(19) United States
(12) Patent Application Publication
(10) Pub. No.: US 2012/0271163 A1
(43) Pub. Date: Oct. 25, 2012

(54) ULTRASONIC MONITORING OF IMPLANTABLE MEDICAL DEVICES

(76) Inventors: Arthur J. Foster, Centerville, MN (US); Binh C. Tran, Minneapolis, MN (US)

(21) Appl. No.: 13/422,507

(22) Filed: Mar. 16, 2012

Related U.S. Application Data
(60) Provisional application No. 61/477,270, filed on Apr. 20, 2011.

(51) Int. Cl. A61B 8/00; A61N 1/05

(52) U.S. Cl. 600/424; 607/116

(57) ABSTRACT

Systems and methods for ultrasonically monitoring implantable medical devices are disclosed. An ultrasonic monitoring system includes an ultrasonic transmitter that transmits an ultrasonic wave into the body, an implantable medical device including at least one ultrasonic reflecting unit configured for reflecting a portion of the ultrasonic wave, and an ultrasonic imaging monitor configured to receive a reflected portion of the ultrasonic wave and produce an ultrasonic image of the implantable medical device within the body. The ultrasonic reflecting unit can include an echogenic fluid medium that reflects a portion of the ultrasonic wave received from the ultrasonic transmitter. The ultrasonic reflecting units can be positioned at various locations on the device to produce localized areas of increased echogenicity, which can be used by the implanting physician to gauge the location of the device within the body.

### CROSS REFERENCE TO RELATED APPLICATIONS

This application claims priority to U.S. Application No. 61/477,270, filed on Apr. 20, 2011, which is incorporated herein by reference in its entirety for all purposes.

### TECHNICAL FIELD

The present invention relates to techniques for monitoring implantable medical devices. More specifically, the present invention pertains to systems and methods for ultrasonically monitoring implantable medical devices within the body.

### BACKGROUND

The implantation of implantable medical devices (IMDs) is often accomplished using x-ray fluoroscopy techniques in which a fluoroscopic monitor is used to visualize the location of the IMD within the body. In the delivery of an implantable cardiac lead, for example, a radiopaque marker located on the distal end of the lead may be used to visualize the lead on a fluoroscopic monitor, allowing the physician to gauge the location and positioning of the lead within the heart and/or cardiac vessels leading into or from the heart. In some cases, portions of the introducer catheter, guidewire, and/or stylet used as part of the lead delivery system may also be fluoroscopically monitored to gauge the location and positioning of the lead delivery system within the body. Although fluoroscopic imaging techniques are widely used in the delivery of implantable leads, such techniques subject the patient to ionizing radiation during the implantation procedure. Furthermore, the equipment required to fluoroscopically image IMDs such as leads is often expensive and requires a significant amount of dedicated space at the location where the procedure is to be performed.

Ultrasonic imaging techniques that rely on acoustic energy instead of ionizing radiation have been introduced as a less invasive means for visualizing IMDs within the body. The visualization of IMDs using ultrasound is typically based on the detection of signals emanating from the device within a surrounding medium such as cardiac tissue. However, many IMDs to be visualized do not include an active ultrasound transmitter, but instead rely upon the reflection of ultrasonic waves impinging upon the device. Enhancement of these reflections increases the ability to visualize such IMDs.

### SUMMARY

The present invention pertains to systems and methods for ultrasonically monitoring implantable medical devices within the body. In Example 1, an implantable medical lead comprises: a lead body having a proximal section and a distal section; and at least one ultrasonic reflecting unit configured for increasing the echogenicity of the lead body when subjected to ultrasonic energy, the ultrasonic reflecting unit including an echogenic fluid medium adapted to reflect a portion of the ultrasonic energy.

In Example 2, the implantable medical lead according to Example 1, wherein the echogenic fluid medium comprises one or more microscopic cavities adapted to oscillate and emit ultrasonic waves in response to the ultrasonic energy.

In Example 3, the implantable medical lead according to Example 2, wherein the ultrasonic reflecting unit comprises at least one tubular member, and wherein the microscopic cavities are embedded within the tubular member.

In Example 4, the implantable medical lead according to any of Examples 1-3, wherein the ultrasonic reflecting unit comprises at least one air-filled well.

In Example 5, the implantable medical lead according to any of Examples 1-4, wherein the lead further comprises a conductor coil electrically coupled to an electrode, and wherein the ultrasonic reflecting unit comprises a helically-shaped coil or ribbon radially disposed about the coil.

In Example 6, the implantable medical lead according to any of Examples 1-5, wherein the lead further comprises a passive lead fixation element, and wherein the echogenic fluid medium is disposed within an interior space of the fixation element.

In Example 7, the implantable medical lead according to any of Examples 1-6, wherein passive fixation element includes an interior space configured to receive an echogenic fluid medium comprising a solution of gas-filled microbubbles.

In Example 8, the implantable medical lead according to any of Examples 1-7, wherein the lead body includes a fluid conduit in communication with the cavity and an external source of gas-filled microbubbles.

In Example 9, the implantable medical lead according to any of Examples 1-8, wherein the at least one ultrasonic reflecting unit comprises a first ultrasonic reflecting unit located at a tip of the lead and at least one additional ultrasonic reflecting unit located on the lead body proximal to the first ultrasonic reflecting unit.

In Example 10, the implantable medical lead according to Example 9, wherein the ultrasonic reflecting units are spaced apart from each other along a length of the lead such that, when visualized using an ultrasonic imaging monitor, each reflecting unit produces a corresponding reflective region on the monitor.

In Example 11, the implantable medical lead according to Example 9, wherein the ultrasonic reflecting units are spaced apart from each other along a length of the lead such that, when visualized using an ultrasonic imaging monitor, the reflecting units produce a continuous reflective region on the monitor.

In Example 12, a system for ultrasonically monitoring an implantable medical device within a body comprises: an ultrasonic transmitter configured for transmitting an ultrasonic wave into the body; an implantable medical device including at least one ultrasonic reflecting unit configured for enhancing a reflected portion of the ultrasonic wave, the reflecting unit including an echogenic fluid medium; and an ultrasonic imaging monitor configured to receive the reflected portion of the ultrasonic wave and generate an ultrasonic image of the implantable medical device within the body.

In Example 13, the ultrasonic monitoring system according to Example 12, wherein the echogenic fluid medium comprises one or more microscopic cavities adapted to oscillate and emit ultrasonic waves in response to the ultrasonic wave.

In Example 14, the ultrasonic monitoring system according to Example 12 or 13, wherein the ultrasonic wave is transmitted at an interrogation frequency, and where the ultrasonic reflecting units are configured to transmit the interrogation frequency and a harmonic of the interrogation frequency.

In Example 15, the ultrasonic monitoring system according to any of Examples 12-14, wherein the ultrasonic reflecting unit comprises at least one air-filled well.

In Example 16, the ultrasonic monitoring system according to any of Examples 12-15, wherein two or more ultrasonic reflecting units are spaced apart from each other along a length of the lead such that each reflecting unit produces a corresponding echogenic region on the monitor.

In Example 17, the ultrasonic monitoring system according to any of Examples 12-16, wherein two or more ultrasonic reflecting units are spaced apart from each other along a length of the lead such that the reflecting units produce a continuous echogenic region on the monitor.

In Example 18, the ultrasonic monitoring system according to any of Examples 12-17, wherein the lead further comprises a conductor coil electrically coupled to an electrode, and wherein the ultrasonic reflecting unit comprises a helically-shaped coil or ribbon radially disposed about the coil.

In Example 19, the ultrasonic monitoring system according to any of Examples 12-18, wherein the lead further comprises a passive lead fixation element, and wherein the echogenic fluid medium is disposed within an interior space of the fixation element.

In Example 20, a method for ultrasonically monitoring an implantable medical lead within a body comprises: inserting an implantable medical lead into a body, the lead including a lead body having a proximal section, a distal section, and a fluid conduit extending between the proximal and distal sections; coupling a solution of gas-filled microbubbles to the fluid conduit and injecting the solution into one or more cavities located within the distal section of the lead body, the microbubbles configured to oscillate when subjected to ultrasonic energy; transmitting an ultrasonic wave into the body; and generating an image of the implantable medical lead within the body based on a reflected portion of the transmitted ultrasonic wave.

While multiple embodiments are disclosed, still other embodiments of the present invention will become apparent to those skilled in the art from the following detailed description, which shows and describes illustrative embodiments of the invention. Accordingly, the drawings and detailed description are to be regarded as illustrative in nature and not restrictive.

### BRIEF DESCRIPTION OF THE DRAWINGS

FIG. 1 is a schematic diagram of a system for ultrasonically monitoring an implantable medical device inserted into a patient's body;

FIG. 2 is a perspective view showing a passive fixation lead including one or more ultrasonic reflecting units for use with an ultrasonic monitoring system;

FIG. 3 is a schematic view showing an ultrasonic reflecting unit incorporated on a portion of an implantable lead comprising microscopic cavities embedded in a material of the lead for enhancing the echogenicity of the lead;

FIG. 4 is a schematic view showing an illustrative lead fixation element comprising a material embedded with microscopic cavities;

FIG. 5 is a schematic view showing another illustrative lead fixation element including one or more ultrasonic reflecting units;

FIG. 6 is a schematic view showing another ultrasonic reflecting unit located within the distal section of an implantable lead including a passive fixation element configured to receive an injected solution of gas-filled microbubbles;

FIG. 7 is a schematic view showing the distal section of another implantable lead including a passive fixation element configured to receive an injected solution of microbubbles;

FIG. 8 is a schematic view showing an ultrasonic reflecting unit located on the distal section of another implantable lead;

FIG. 9 is a schematic view showing a portion of another implantable lead including a helical coil or ribbon configured to enhance the echogenicity of the lead;

FIG. 10 is a schematic view showing a portion of another implantable lead including a number of ring-shaped collars configured to enhance the echogenicity of the lead;

FIG. 11 is a schematic view showing a distal section of another implantable lead including multiple ultrasonic reflecting units configured to enhance the echogenicity of the lead;

FIG. 12 is a schematic view showing a distal section of another implantable lead including multiple ultrasonic reflecting units configured to enhance the echogenicity of the lead; and

FIG. 13 is a schematic view showing a distal section of another implantable lead including a continuous ultrasonic reflecting unit configured to enhance the echogenicity of the lead.

While the invention is amenable to various modifications and alternative forms, specific embodiments have been shown by way of example in the drawings and are described in detail below. The intention, however, is not to limit the invention to the particular embodiments described. On the contrary, the invention is intended to cover all modifications, equivalents, and alternatives falling within the scope of the invention as defined by the appended claims.

### DETAILED DESCRIPTION

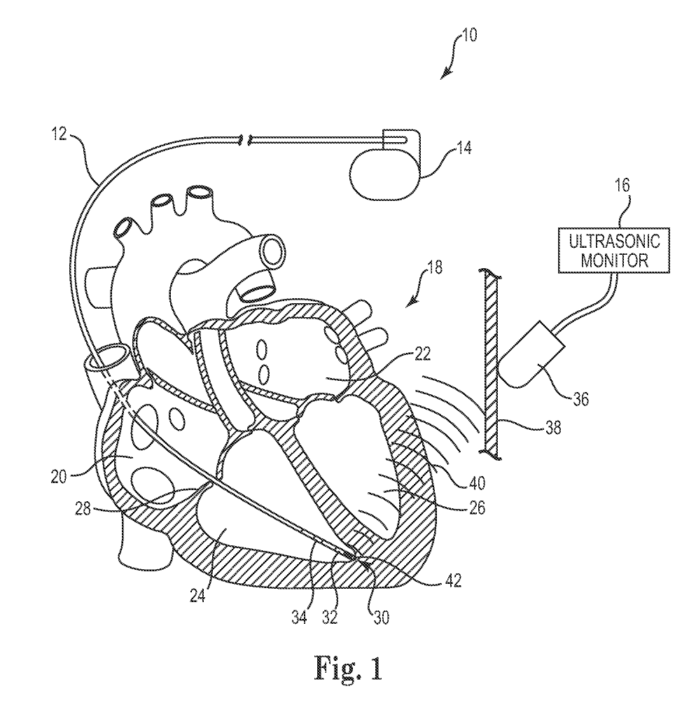

FIG. 1 is a schematic diagram of a system 10 for ultrasonically monitoring an implantable medical device inserted into a patient's body in accordance with an illustrative embodiment. The system 10, illustratively a cardiac lead system for providing cardiac rhythm management or cardiac disease management, includes an implantable lead 12 coupled to a pulse generator 14, and an ultrasonic imaging monitor 16 that can be used to ultrasonically visualize the lead 12 within the body. During lead delivery, the ultrasonic imaging monitor 16 may be used to guide the lead 12 to a target implantation region in or near a patient's heart 18, which includes a right atrium 20, a left atrium 22, a right ventricle 24, and a left ventricle 26. In the embodiment of FIG. 1, for example, the lead 12 comprises a right ventricle lead that may directed through the right atrium 20, through the tricuspid valve 28, and into the apex 30 of the right ventricle 24 using the ultrasonic imaging monitor 16 to ultrasonically visualize the location of the lead 12 in real-time.

Although for purposes of illustration the lead 12 is shown inserted into the right ventricle 24 of the heart 18, the system 10 may be used as an aid to implant the lead 12 at other target regions in or near the heart 18 and/or to implant multiple leads in or near the heart 18. In some embodiments, for example, the system 10 may be used to implant a lead in the right atrium 20, the left atrium 22, the left ventricle 26, or in a coronary vessel leading into or from the heart 18. Other types of cardiac leads such as epicardial or endocardial leads may also be visualized using the system 10. Moreover, while the system 10 is described with respect to cardiac leads, in other embodiments the ultrasonically visible leads and lead structures described herein can be used with other types of implantable leads such as implantable neurostimulation leads. In addition, the different structures described herein can also be used in conjunction with other IMDs used for providing other types of therapy within the body. For example, the echogenic features discussed herein can be incorporated into an miniature leadless device such as an injectible microstimulator.

In some embodiments, the ultrasonic imaging monitor 16 can be used in addition to fluoroscopy techniques to enhance visualization of the lead 12 within the body. In certain embodiments, for example, the ultrasonic imaging monitor 16 can be used as the primary means to visualize the lead 12, and a fluoroscopic monitor may serve as a backup in the event ultrasonic imaging of the lead 12 is not possible, or in the event additional visualization is desired. In other embodiments, the ultrasonic imaging monitor 16 may serve as an alternative to fluoroscopy. Unlike fluoroscopy, which subjects the patient to ionizing radiation during the implantation procedure, the use of ultrasonic energy to visualize IMDs within the body can be performed for extended periods of time without subjecting the patient to radiation, and can be used at locations where fluoroscopy equipment is unavailable.

In the embodiment of FIG. 1, the lead 12 includes one or more cardiac pace/sense electrodes 32, 34 for sensing electrical measurements within the patient's heart 18 and/or for delivering pacing pulses and/or defibrillation energy to the heart 18. Once implanted at a desired location in or near the heart 18, the lead 12 can be connected to the pulse generator 14, which provides electrical stimulation pulses to the lead electrodes 32, 34 and, in some cases, defibrillation energy to the electrodes 32, 34. In certain embodiments, for example, the electrodes 32, 34 may be provided as part of a cardiac lead 12 used to treat bradycardia, tachycardia, or other cardiac arrhythmias. During normal operation, the lead 12 can be configured to convey electrical signals between the pulse generator 14 and the heart 18. For example, in those embodiments where the pulse generator 14 is a pacemaker, the lead 12 can be utilized to deliver electrical therapeutic stimulus for pacing the heart 18. In other embodiments in which the pulse generator 14 is an implantable cardiac defibrillator, the lead 12 can be utilized to deliver electric shocks to the heart 18 in response to an event such as an arrhythmia. In some embodiments, the pulse generator 14 includes both pacing and defibrillation capabilities.

An ultrasonic transducer 36 in communication with the ultrasonic imaging monitor 16 can be applied or attached to the surface of the patient's skin 38, and is configured to generate ultrasonic waves 40 that are transmitted into the patient's body towards the general location of the lead 12. In a passive lead imaging system, for example, the ultrasonic transducer 36 transmits ultrasonic waves through the body that impinge upon the lead and produce a reflected ultrasonic wave, which can be received and analyzed to produce a real-time image of the lead within the body. In some embodiments, the excitation frequency of the ultrasonic transducer 36 is at or between about 1 MHz to 5 MHz, and more specifically, about 3 MHz. As is discussed further herein, one or more ultrasonic reflecting units on the lead 12 serve to improve the echogenicity of the lead 12 when subjected to ultrasonic waves 40 from the transducer 36. In some embodiments, the one or more ultrasonic reflecting units are configured to enhance the visualization of the lead 12 using standard ultrasonic imaging techniques (e.g., B-mode, M-mode, or Doppler mode) while being substantially transparent to fluoroscopy. In other embodiments, the one or more ultrasonic reflecting units are configured to enhance the visualization of the lead 12 using advanced ultrasonic imaging techniques such as harmonic imaging.

When visualized using the ultrasonic imaging monitor 16, the increased echogenicity from the ultrasonic reflecting units serves to increase the visibility (e.g., contrast) of the lead 12 relative to the surrounding body tissue. The ultrasonic reflecting units are adapted to improve echogenicity without affecting the desired mechanical and electrical characteristics of the lead 12. In some embodiments, the location of the ultrasonic reflecting units may impart localized areas of increased echogenicity to specific regions of the lead 12 to facilitate identification of those regions within the body relative to other anatomical features. In one embodiment, for example, the presence of an ultrasonic reflecting unit on the lead tip 42 may be used to facilitate identification of the tip 42 during lead delivery. Ultrasonic reflecting units can also be placed at other locations of the lead 12 to enhance ultrasonic visualization using the ultrasonic imaging monitor 16.

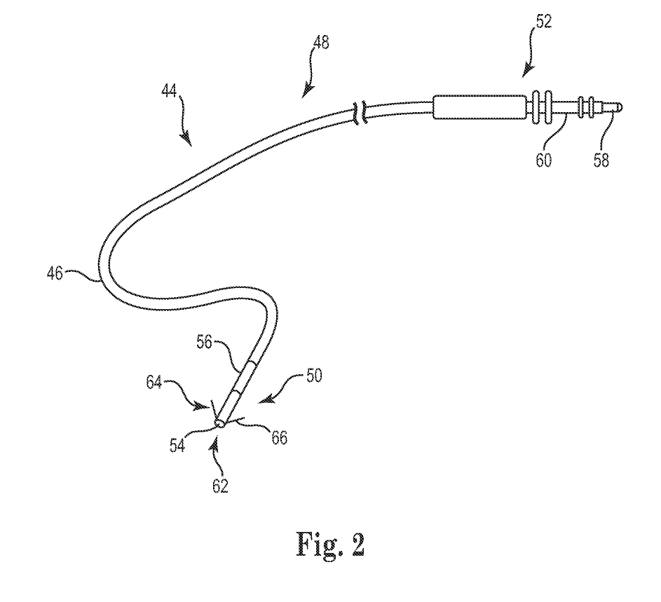

FIG. 2 is a perspective view showing an illustrative passive fixation lead 44 including one or more ultrasonic reflecting units for use with an ultrasonic monitoring system. The lead 44 can comprise, for example, an implantable cardiac lead that can be used in conjunction with the ultrasonic imaging system 10 of FIG. 1. In the embodiment of FIG. 2, the lead 44 includes a lead body 46 having a proximal section 48 and a distal section 50. The proximal section 48 of the lead 44 includes a terminal end connector 52 that can be coupled to a pulse generator, which supplies electrical stimulus energy to a number of lead electrodes 54,56 on the distal section 50 of the lead 44. The terminal end connector 52 includes a terminal pin contact 58 and terminal ring contact 60, which are electrically connected to electrodes 54 and 56, respectively, via corresponding electrical conductors located within the lead body 46. The terminal pin contact 58, for example, is electrically connected to a tip electrode 54 located at the distal end 62 of the lead body 46. The terminal ring contact 60, in turn, is electrically connected to a ring electrode 56 located proximal to the tip electrode 54. A passive fixation element 64 including a number of fixation tines 66 are configured for use in securing the lead 44 to adjacent body tissue once positioned at a desired location within the body.

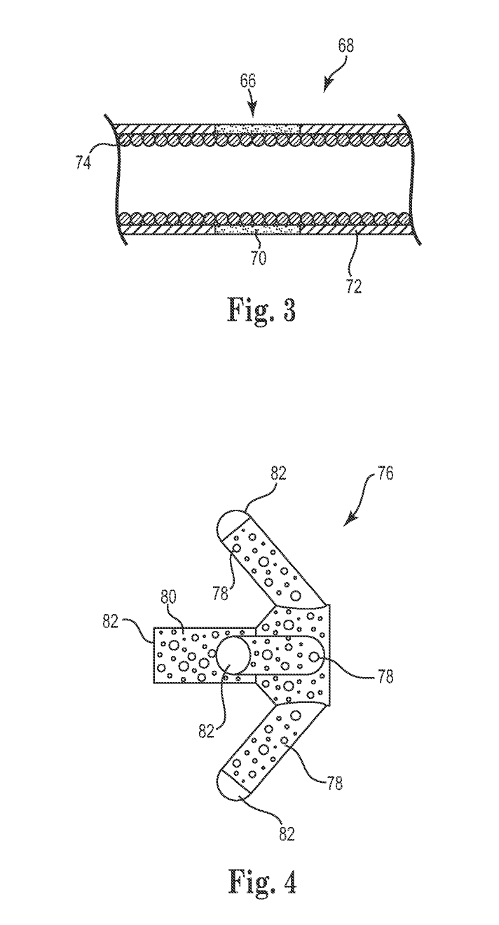

FIG. 3 is a schematic view showing an ultrasonic reflecting unit 66 incorporated on a portion of an implantable lead 68, the ultrasonic reflecting unit 66 comprising microscopic cavities 70 embedded in a material of the lead 68 for enhancing the echogenicity of the lead 68. The lead 68 can comprise, for example, a cardiac or neurostimulation lead that can be visualized using the ultrasonic monitoring system 10 of FIG. 1. As shown in FIG. 3, the lead 68 includes a tubular member 72 radially disposed about an electrical conductor coil 74. The tubular member 72 and conductor coil 74 can comprise, for example, part of an electrical conductor coil assembly disposed within an implantable lead for use in electrically connecting one or more lead electrodes to a pulse generator. In other embodiments, the tubular member 72 and conductor coil 74 can be utilized in other types implantable leads or in other IMDs that utilize electrical conductors for supplying electrical energy.

In the embodiment of FIG. 3, the tubular member 72 comprises an electrically non-conductive polymeric material, which serves as an insulator for the conductor coil 74. Microscopic cavities 70, embedded in a portion of the tubular member 72, form an ultrasonic reflecting unit 66, which increases the echogenicity of this portion of the lead 68 without altering the desired mechanical and electrical characteristics of the coil 74. The microscopic cavities 70 can be embedded in the polymeric tubular member 72 along all or a portion of the coil length, and is configured to increase the echogenicity of the lead 68 under ultrasonic visualization. The microscopic cavities 70 can also be embedded in multiple small sections, forming multiple ultrasonic reflecting units 66 at other locations on the lead 68 for increasing the visibility of other portions of the lead 68 when subjected to ultrasonic energy. In some embodiments, the portion of the tubular member 72 with the ultrasonic reflecting unit 66 may be comprised of a polymer different from the remaining portion of the tubular member 72.

In some embodiments, the microscopic cavities 70 are microspheres configured to resonate at or near the excitation frequency of the ultrasonic waves transmitted from the ultrasonic transducer. In one embodiment, the microspheres are spherical occlusions filled with air, gas, or other fluid and suspended within the polymeric tubular member 72. When the ultrasonic waves impinge upon the tubular member 72, the microscopic cavities 70 are configured to oscillate and emit an enhanced reflected ultrasonic wave that can be sensed by the ultrasonic transducer operating in a receive mode. The microscopic cavities 70 act as harmonic pulsators to accentuate the acoustic signal reflected from the lead 68, which results in improved visual contrasting of the lead 68 on the ultrasonic imaging monitor. In some embodiments, the microscopic cavities 70 can be configured to radiate a reflected acoustic signal comparable to that of a much larger physical structure that does not include an echogenic enhancement without altering the desired size and performance characteristics of the lead 68. The strength of the enhanced reflected signals is dependent in part on the difference in acoustic impedance of the ultrasonic reflecting unit 66 and the surrounding medium, as well as the size, shape, density, and fluid (gas) content of the microscopic cavities 70.

In some embodiments, the signal strength enhancement provided by the ultrasonic reflecting unit 66 and in particular the microscopic cavities 70, can be optimized to a specific imaging frequency or range of frequencies by changing the distribution of cavity radii, the density of the gas within the cavities, the outer surface or shell of the cavity (e.g., the material of the tubular member 72), as well as other parameters. An example microscopic cavity 70 suitable for use in cardiac lead imaging comprises a 1 micron sized air-filled bubble having a resonance frequency at or near about 3 MHz.

The microscopic cavities 70 can be incorporated into other polymeric structures on the lead 68, forming additional ultrasonic reflecting units on the lead 68 that are visible and identifiable in an ultrasound image. For example, in some embodiments, an ultrasonic reflecting unit is located on the tip of a passive fixation lead. In some embodiments, the ultrasonic reflecting units are comprised of microscopic cavities embedded within portions of the lead 68 during the manufacturing process, and are configured to remain in the lead 68 permanently to facilitate visualization throughout the life of the lead 68. In other embodiments, the ultrasonic reflecting unit comprise transient microscopic cavities in the form of microbubble solutions injected into lead 68 prior to and/or during implantation of the lead 68 within the body.

Other echogenic features can also be incorporated into the tubular member 72 and/or other lead components for increasing the echogenicity of the lead 68. In one embodiment, for example, specifically patterned air-filled wells may be embedded into the tubular member 72 which, similar to the microscopic cavities 70, function as an ultrasonic reflecting unit to increase the echogenicity of the tubular member 72 under ultrasonic imaging.

FIG. 4 is a schematic view showing an illustrative lead fixation element 76 comprising a material with embedded microscopic cavities 78 forming an ultrasonic reflecting unit to enhance the echogenicity of the tip of an implantable lead. In one embodiment, the microscopic cavities 80 comprise microspheres embedded in the lead tip similar to the microscopic cavities described herein, but may differ in size, shape, density, and so forth due to the more rigid polymer typically used in lead tips than that used in the lead insulation. The lead fixation element 76 can comprise, for example, a passive fixation element used for securing an implantable lead to adjacent body tissue (e.g., cardiac tissue) within a patient's body. As shown in FIG. 4, the fixation element 76 comprises a base 80 and a number of fixation tines 82. In the embodiment of FIG. 4, the base 80 and tines 82 each include a number of gas-filled microspheres 80 configured to increase the echogenicity of the lead tip when visualized in conjunction with an ultrasonic imaging monitor. In other embodiments, only selective portions of the fixation element 76 (e.g., the fixation tines 82) include gas-filled microspheres 80. As with other embodiments discussed herein, the gas-filled microspheres 80 are configured to oscillate and emit an ultrasonic wave that can be sensed by the ultrasonic transducer operating in a receive mode. Other ultrasonic reflecting units can be located on or within the fixation element 76 for increasing the visibility of the lead tip under ultrasonic imaging.

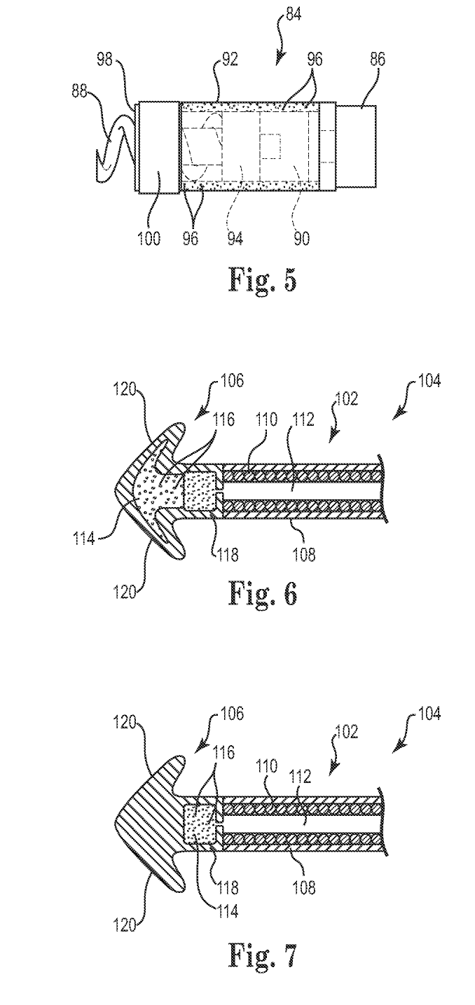

FIG. 5 is a schematic view showing another illustrative lead fixation element 84 including one or more ultrasonic reflecting units for use with an ultrasonic monitoring system. The fixation element 84 can comprise, for example, an active lead fixation element coupled to a lead body 86 for securing an implantable lead to adjacent body tissue within a patient's body. In the embodiment of FIG. 5, the fixation element 84 includes a fixation helix 88 adapted to rotate and translate out from within an interior space 90 of a collar 92. The fixation helix 86 is coupled proximally to a base 94, which, in turn, is rotatably coupled to an elongate shaft (not shown) that extends through the interior of the lead body 86, and which can be manipulated by the implanting physician from a location outside of the patient's body. During implantation, the physician may rotate the base 94 in either a clockwise or counterclockwise direction via the elongate shaft, causing the fixation helix 88 to either extend or retract from within the interior space 90 of the collar 92.

In the embodiment of FIG. 5, the collar 90 comprises a polymeric tube or sheath including a number of microscopic air cavities 96. In some embodiments, the microscopic air cavities 96 may be embedded within the collar 90 along the entire length of the collar 90. In other embodiments, the microscopic air cavities 96 may be embedded in only a portion of the collar 90 such as at a distal end 98 of the collar 90. The microscopic air cavities 96 can also be embedded in other portions of the fixation element 84 such as the fixation helix 88 and base 94. Other echogenic features or ultrasonic reflecting units can be located on or within the fixation element 84 for increasing the visibility of the element 84 under ultrasonic imaging. In some embodiments, the microscopic air cavities 96 can be used in conjunction with other features for enhancing the visibility of the lead under fluoroscopy. In certain embodiments, for example, the microscopic air cavities 96 can be used in conjunction with a radiopaque marker 100 located at or near the distal end 98 of the collar 92 to enhance lead visibility using fluoroscopy and ultrasonic imaging.

FIG. 6 is a schematic view showing another ultrasonic reflecting unit located within the distal section 102 of an implantable lead 104 including a passive fixation element 106 configured to receive an injected solution of gas-filled microbubbles. As shown in FIG. 6, the lead 104 includes a lead body 108 that houses a conductive coil 110. An interior lumen 112 within lead body 108 is configured to receive a small amount of a microbubble solution, such as an ultrasonic contrast agent, to enhance the echogenicity of the fixation element 106 when subjected to ultrasonic energy. In the embodiment of FIG. 6, the interior lumen 112 is in fluid communication with an interior cavity 114 disposed within the interior of the fixation element 106. Prior to implantation, a solution of gas-filled microbubbles 116 is fluidly coupled to the lumen 112, and is injected into the cavity 114. During ultrasonic visualization, the microbubble-filled cavity 114 functions as an ultrasonic reflecting unit to enhance the visibility of the distal lead section 102, and in particular the lead tip, under ultrasonic imaging. Since microbubbles contained within a solution are typically unstable and will eventually dissolve, injections of the solution can be made periodically throughout the implantation procedure to replenish or increase the supply of bubbles in the cavity 114, if desired.

In the embodiment, the interior cavity 114 is located within both a base 118 and within a portion of each of the fixation tines 120. The location of the interior cavity 114 can vary, however. In one embodiment shown in FIG. 7, for example, the interior cavity 114 is located within only the base 118 of the fixation element. In another embodiment, the interior cavity 114 is located within only the fixation tines 118. The interior cavity 114 can also be located in other sections of the lead 104. In some embodiments, fluid channels within the lead 104 provide a conduit to transport microbubbles to cavities within the tubular insulation of the lead 104. In some embodiments, multiple interior cavities 114 adapted to receive a solution of gas-filled microbubbles can be used to enhance visibility at other locations on the lead 104.

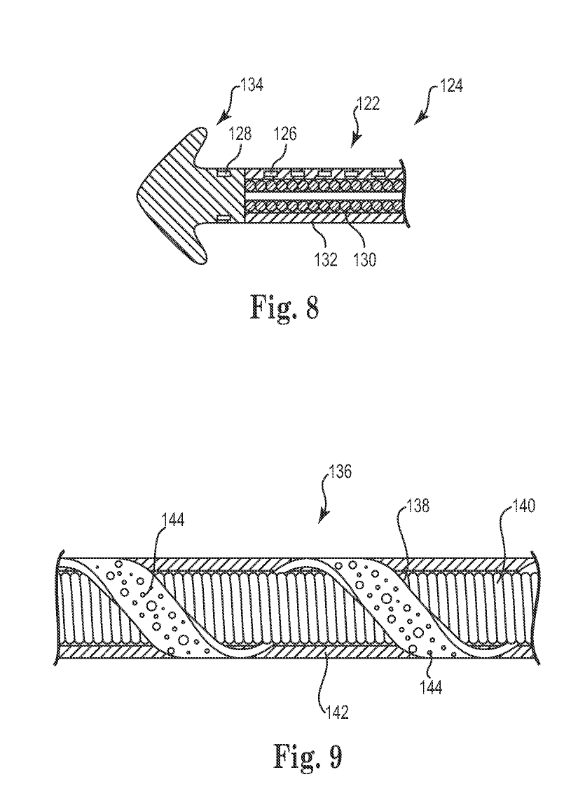

FIG. 8 is a schematic view showing an ultrasonic reflecting unit located on the distal section 122 of another implantable lead 124, the ultrasonic reflecting unit including one or more air-filled wells 126, 128 configured to enhance the echogenicity of the lead 124. As shown in FIG. 8, the lead 124 includes a conductor coil 130 and a lead body 132 comprising a non-conductive polymeric material. In the embodiment of FIG. 8, the ultrasonic reflecting unit is comprised of multiple, small air wells 126, 128 that function cooperatively to increase ultrasonic reflection. In another embodiment, the ultrasonic reflecting unit is comprised of a single, larger air well disposed circumferentially within the polymeric material around the lead body 132.

One or more air-filled wells 126 located within the lead body 132 along the length of the lead 124 are configured to enhance the echogenicity of the lead 124 under ultrasonic imaging. In some embodiments, and as shown in FIG. 8, the air-filled wells 126 are positioned eccentrically on one side of the of the lead body 132, allowing the implanting physician to more accurately gauge the orientation of the lead 124 relative to other anatomical structures. In other embodiments, the air-filled wells 126 may be spaced evenly about the circumference of the lead body 132. Ultrasonic reflecting units comprised of air-filled wells may also be placed at other locations on the lead 124 to increase the echogenicity of the lead 124 at other locations. In some embodiments, for example, one or more air-wells 128 located within the distal tip 134 of the lead 124 may be used to increase the echogenicity of the tip 134.

During ultrasonic imaging, the air within each of the wells 126, 128 reflects the ultrasonic waves without compromising the flexibility, electrical, and other desired performance characteristics of the lead 124. The air within each of the wells 126, 128 has an acoustic impedance that is several orders of magnitude different from that of the surrounding body tissue. This impedance mismatch serves to reflect the incident ultrasonic waves which, when visualized via an ultrasonic imaging monitor, result in an area of contrast that can be seen on the monitor. The ultrasound wave can also exert pressure on the air in the well, causing the well to act like a resonator and emit larger reflections. The size and shape of the air-filled wells 126, 128 can be configured so as to adjust the amount of reflectivity provided by the lead 124. Example shapes for the air-filled wells 126, 128 can include, but are not limited to, spherical, rectangular, and toroidal shaped wells.

For some ultrasonic reflecting units comprising echogenic structures such as microscopic cavities, microspheres, and air-filled wells, a greater contrast of the reflective structure relative to the surrounding body tissue can be observed in a harmonic (e.g., a second harmonic) of the excitation imaging frequency used by the ultrasonic imaging monitor. In some cases, these echogenic structures emit harmonic and subharmonic oscillations whereas body tissues surrounding the device can only reflect the excitation signal. In those embodiments in which the ultrasonic reflecting unit produces signals at harmonics of the imaging frequency, advanced imaging modes on an ultrasonic imaging monitor can be exploited to further enhance the visualization of the device.

FIG. 9 is a schematic view showing a portion of another implantable lead 136 including a helical coil or ribbon 138 configured to enhance the echogenicity of the lead 136. The lead 136 includes a conductor coil 140 and an insulative body 142 comprising a polymeric material such as silicone or polyurethane.

In the embodiment of FIG. 9, the helical coil or ribbon 138 comprises a material embedded with gas-filled microbubbles 144, which serve to increase the echogenicity of the lead 136 under ultrasonic imaging. In some embodiments, the helical coil or ribbon 138 is radially disposed about the conductor coil 140 along the entire length of the coil 140. In other embodiments, the helical coil or ribbon 138 is radially disposed about the conductor coil 140 along only a portion of the coil length. In other embodiments, the helical coil or ribbon 138 is embedded within the insulative body 142 of the lead 136. During ultrasonic imaging, the gas-filled microcavities 144 increase the echogenicity of the lead 136 without compromising the flexibility, electrical, and other desired performed characteristics of the lead 136.

In certain embodiments, the material forming the helical coil or ribbon 138 comprises a polymeric or metallic material having an acoustic impedance significantly different from the blood and other body tissue surrounding the lead 136 as well as the materials used in forming the conductor coil 140 and insulative body 142. Example materials that can be used for forming the helical coil or ribbon 140 include titanium or high density polyvinylidene fluoride (PVDF). The difference in acoustic impedance due to the material properties of the helical coil or ribbon 138 relative to the surrounding body tissue and other lead components also serves to increase the echogenicity of the lead 136 under ultrasonic imaging.

Other structural features on the lead 136 can also serve as ultrasonic reflecting units to increase the echogenicity of the lead 136 in addition to, or in lieu of, the helical coil or ribbon 138. Example structural features that can be employed to increase the echogenicity can include collars, rings, or bands having an acoustic impedance that is significantly different from that of the surrounding body tissue. In some embodiments, microbubbles, microspheres, and/or air-filled wells can also be utilized in addition to the material characteristics of the structure to increase the echogenicity of the lead 136.

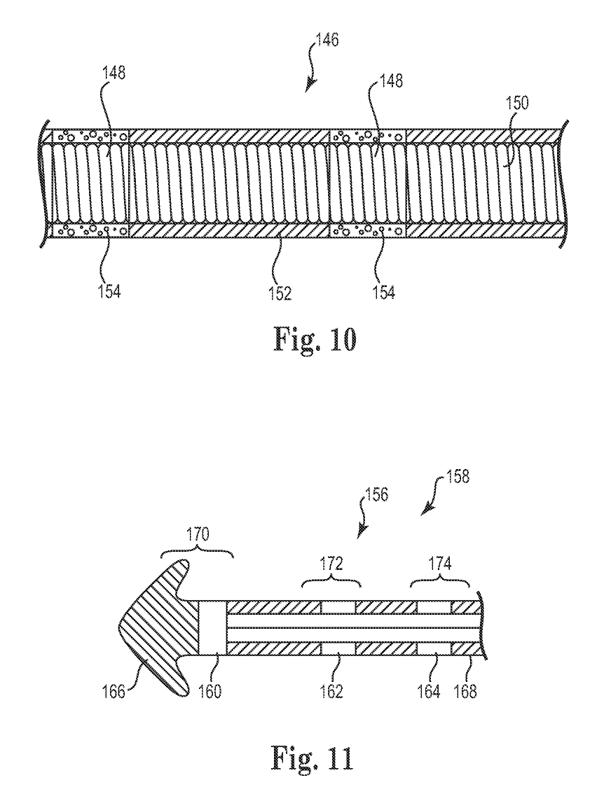

FIG. 10 is a schematic view showing a portion of another implantable lead 146 including a number of ring-shaped collars 148 configured to enhance the echogenicity of the lead 146. As shown in FIG. 10, the lead 146 includes a conductor coil 150 and an insulative body 152 comprising a non-conductive material such as silicone or polyurethane.

In the embodiment of FIG. 10, each of the collars 148 comprise a material embedded with gas-filled microscopic cavities 154. In other embodiments, the collars 148 do not contain microscopic cavities. In some embodiments, the material forming the collars 148 has an acoustic impedance that is significantly different from the blood and other tissue surrounding the lead 146.

One or more of the ultrasonic reflecting units described herein can be positioned on an implantable device to produce localized areas of increased echogenicity relative to other regions on the device. In some embodiments, multiple ultrasonic reflecting units can be positioned on the device such that, when visualized using an ultrasonic imaging monitoring, the reflecting units appear as a single, contiguous reflecting region on an ultrasonic image. Some echogenic structures such as resonating bubbles and cavities, for example, effectively reflect over an area that is substantially larger than their physical size when oscillations are induced. In another embodiment, multiple smaller ultrasonic reflecting units may be used in place of a single larger unit such that, when imaged with an ultrasonic imaging monitor, the signal is intentionally diffuse to allow identification of anatomical structures in the vicinity of the device. The combination of structure type, physical size, shape, and material configuration of the reflective feature may be selected based on the enhancement in the ultrasound image desired.

FIG. 11 is a schematic view showing a distal section 156 of another implantable lead 158 including multiple ultrasonic reflecting units 160, 162, 164 configured to enhance the echogenicity of the lead 158 under ultrasonic imaging. In the embodiment of FIG. 11, a first ultrasonic reflecting unit 160 disposed within a distal lead tip 166 of the lead 158 is configured to enhance the echogenicity of the lead 158 at the tip 166. A second number of ultrasonic reflecting units 162, 164 disposed proximally of the first ultrasonic reflecting unit 160, in turn, are disposed within an insulative body 168 of the lead 158, and are similarly configured to increase the echogenicity of the lead 158 along the lead body 168.

The ultrasonic reflecting units 160, 162, 164 can comprise a material embedded with microscopic cavities, injected microbubbles, and/or air-filled wells. The ultrasonic reflecting units 160, 162, 164 can also comprise reflective structures such as a collar or helical shaped coil having an acoustic impedance that is significantly different from the surrounding body tissue. In some embodiments, the ultrasonic reflecting units 160, 162, 164 can each include a combination of echogenic features.

The ultrasonic reflecting units 160, 162, 164 can be spaced apart from each other a distance such that, when visualized using an ultrasonic imaging monitor, the reflecting units 160, 162, 164 produce a number of discrete reflective regions 170, 172, 174 on the lead 158 that can be used by the implanting physician to identify certain lead features on the ultrasonic image such as the lead tip and electrodes. In certain embodiments, for example, the ultrasonic reflecting units 160, 162, 164 can be spaced apart from each other a distance of about 20 mm along the length of the lead body 168, producing three discrete reflective regions 170, 172, 174 on the ultrasonic image. The distance between each reflecting unit 160, 162, 164 may differ, however, depending on the structural and material characteristics of the reflecting units 160, 162, 164, the location of the device within the body, the characteristics of the imaging system, as well as other factors.

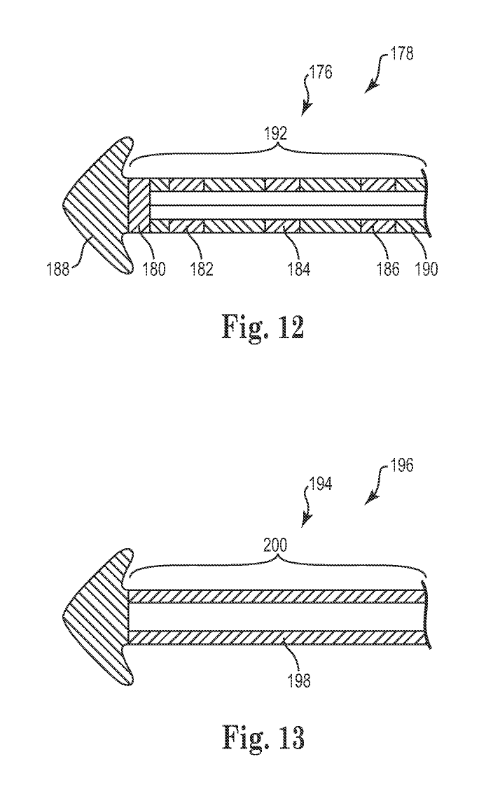

FIG. 12 is a schematic view showing a distal section 176 of another implantable lead 178 including multiple ultrasonic reflecting units 180, 182, 184, 186 configured to enhance the echogenicity of the lead 178 under ultrasonic imaging. In the embodiment of FIG. 12, a first ultrasonic reflecting unit 180 disposed within a distal lead tip 188 is configured to enhance the echogenicity of the lead 178 at the tip 188. A second number of ultrasonic reflecting units 182, 184, 186 disposed proximally of the first ultrasonic reflecting unit 180, in turn, are disposed within an insulative body 190 of the lead 178, and are similarly configured to increase the echogenicity of the lead 178 along the lead body 190.

The ultrasonic reflecting units 180, 182, 184, 186 can comprise a material embedded with microscopic cavities, microbubbles, and/or air-filled wells. The ultrasonic reflecting units 180, 182, 184, 186 can also comprise reflective structures such as a collar or helical shaped coil or ribbon having an acoustic impedance that is significantly different from the surrounding body tissue. In some embodiments, the ultrasonic reflecting units 180, 182, 184, 186 can each include a combination of echogenic features.

The ultrasonic reflecting units 180, 182, 184, 186 can be closely spaced apart from each other such that, when visualized using an ultrasonic imaging monitor, produce a continuous reflective region 192 on the lead 178 that can be used by the implanting physician to determine the location of the lead 178 within the body. In certain embodiments, for example, the echogenic reflecting units 180, 182, 184, 186 can be spaced apart from each other a distance of about 1 mm to 10 mm, and more specifically, about 5 mm along the length of the lead body 190. The spacing between each reflecting unit 180, 182, 184, 186 may differ, however, depending on the structural and material characteristics of the reflecting units 180, 182, 184, 186, the location of the device within the body, the characteristics of the imaging system, as well as other factors.

FIG. 13 is a schematic view showing a distal section 194 of another implantable lead 196 including a continuous ultrasonic reflecting unit 198 configured to enhance the echogenicity of the lead 194 under ultrasonic imaging. In the embodiment of FIG. 13, the ultrasonic reflecting unit 198 forms a portion of the lead body along all or a substantial length of the distal lead section 194. As with other embodiments, the ultrasonic reflecting unit 198 can comprise a material embedded with microscopic cavities, microbubbles, and/or air-filled wells. The ultrasonic reflecting unit 198 can also comprise a reflective structure such as a collar or helical shaped coil or ribbon having an acoustic impedance that is significantly different from the surrounding body tissue. In some embodiments, the ultrasonic reflecting unit 198 can include a combination of echogenic features.

When visualized with an ultrasonic imaging monitor, the ultrasonic reflecting unit 198 produces a continuous reflective region 200 along the distal section 194 of the lead 196 that can be used by the implanting physician to determine the location of the lead 196 within the body.

Various modifications and additions can be made to the exemplary embodiments discussed without departing from the scope of the present invention. For example, while the embodiments described above refer to particular features, the scope of this invention also includes embodiments having different combinations of features and embodiments that do not include all of the described features. Accordingly, the scope of the present invention is intended to embrace all such alternatives, modifications, and variations as fall within the scope of the claims, together with all equivalents thereof.

What is claimed is:

1. An implantable medical lead, comprising:
a lead body having a proximal section and a distal section; and
at least one ultrasonic reflecting unit configured for increasing the echogenicity of the lead body when subjected to ultrasonic energy, the ultrasonic reflecting unit including an echogenic fluid medium adapted to reflect a portion of the ultrasonic energy.

2. The implantable medical lead of claim 1, wherein the echogenic fluid medium comprises one or more microscopic cavities adapted to oscillate and emit ultrasonic waves in response to the ultrasonic energy.

3. The implantable medical lead of claim 2, wherein the ultrasonic reflecting unit comprises at least one tubular member, and wherein the microscopic cavities are embedded within the tubular member.

4. The implantable medical lead of claim 1, wherein the ultrasonic reflecting unit comprises at least one air-filled well.

5. The implantable medical lead of claim 1, wherein the lead further comprises a conductor coil electrically coupled to an electrode, and wherein the ultrasonic reflecting unit comprises a helically-shaped coil or ribbon radially disposed about the coil.

6. The implantable medical lead of claim 1, wherein the lead further comprises a passive lead fixation element, and wherein the echogenic fluid medium is disposed within an interior space of the fixation element.

7. The implantable medical lead of claim 6, wherein passive fixation element includes an interior space configured to receive an echogenic fluid medium comprising a solution of gas-filled microbubbles.

8. The implantable medical lead of claim 6, wherein the lead body includes a fluid conduit in communication with the cavity and an external source of gas-filled microbubbles.

9. The implantable medical lead of claim 1, wherein the at least one ultrasonic reflecting unit comprises a first ultrasonic reflecting unit located at a tip of the lead and at least one additional ultrasonic reflecting unit located on the lead body proximal to the first ultrasonic reflecting unit.

10. The implantable medical lead of claim 9, wherein the ultrasonic reflecting units are spaced apart from each other along a length of the lead such that, when visualized using an ultrasonic imaging monitor, each reflecting unit produces a corresponding reflective region on the monitor.

11. The implantable medical lead of claim 9, wherein the ultrasonic reflecting units are spaced apart from each other along a length of the lead such that, when visualized using an ultrasonic imaging monitor, the reflecting units produce a continuous reflective region on the monitor.

12. A system for ultrasonically monitoring an implantable medical device within a body, the system comprising:
an ultrasonic transmitter configured for transmitting an ultrasonic wave into the body;
an implantable medical device including at least one ultrasonic reflecting unit configured for enhancing a reflected portion of the ultrasonic wave, the reflecting unit including an echogenic fluid medium; and
an ultrasonic imaging monitor configured to receive the reflected portion of the ultrasonic wave and generate an ultrasonic image of the implantable medical device within the body.

13. The ultrasonic monitoring system of claim 12, wherein the echogenic fluid medium comprises one or more microscopic cavities adapted to oscillate and emit ultrasonic waves in response to the ultrasonic wave.

14. The ultrasonic monitoring system of claim 13, wherein the ultrasonic wave is transmitted at an interrogation frequency, and where the ultrasonic reflecting units are configured to transmit the interrogation frequency and a harmonic of the interrogation frequency.

15. The ultrasonic monitoring system of claim 12, wherein the ultrasonic reflecting unit comprises at least one air-filled well.

16. The ultrasonic monitoring system of claim 12, wherein two or more ultrasonic reflecting units are spaced apart from each other along a length of the lead such that each reflecting unit produces a corresponding echogenic region on the monitor.

17. The ultrasonic monitoring system of claim 12, wherein two or more ultrasonic reflecting units are spaced apart from each other along a length of the lead such that the reflecting units produce a continuous echogenic region on the monitor.

18. The ultrasonic monitoring system of claim 12, wherein the lead further comprises a conductor coil electrically coupled to an electrode, and wherein the ultrasonic reflecting unit comprises a helically-shaped coil or ribbon radially disposed about the coil.

19. The ultrasonic monitoring system of claim 12, wherein the lead further comprises a passive lead fixation element, and wherein the echogenic fluid medium is disposed within an interior space of the fixation element.

20. A method for ultrasonically monitoring an implantable medical lead within a body, the method comprising:
inserting an implantable medical lead into a body, the lead including a lead body having a proximal section, a distal section, and a fluid conduit extending between the proximal and distal sections;
coupling a solution of gas-filled microbubbles to the fluid conduit and injecting the solution into one or more cavities located within the distal section of the lead body, the microbubbles configured to oscillate when subjected to ultrasonic energy;
transmitting an ultrasonic wave into the body; and
generating an image of the implantable medical lead within the body based on a reflected portion of the transmitted ultrasonic wave.
